(19) United States
(12) Patent Application Publication
(10) Pub. No.: US 2010/0121223 A1
(43) Pub. Date: May 13, 2010

(54) Clinical Assessment Scales and Methods

(75) Inventors: Sandra L. Friborg, San Mateo, CA (US); Frederick C. Beddingfield, Pacific Palisades, CA (US); Christine Somogyi, Santa Ana, CA (US); John C. Lue, Huntington Beach, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/606,513

(22) Filed: Oct. 27, 2009

Related U.S. Application Data
(60) Provisional application No. 61/109,000, filed on Oct. 28, 2008.

(51) Int. Cl. A61B 5/103

(52) U.S. Cl. 600/587

(57) ABSTRACT

Disclosed herein are scales for assessing eyelash prominence. The scales comprise rows or columns of photographs corresponding to different categories of eyelash prominence.

### CROSS-REFERENCE

This application claims the benefit of and priority to U.S. Provisional Patent Application Ser. No. 61/109,000, filed on Oct. 28, 2008, the entire disclosure of which is incorporated herein by this specific reference.

### FIELD OF THE INVENTION

The present invention provides scales and methods for performing clinical assessment of an individual. Particularly, the present invention provides reliable scales to effectively rate eyelash prominence.

### BACKGROUND OF THE INVENTION

Eyelash loss or decreasing prominence of eyelashes has been associated with some disease states including endocrine and genetic abnormalities as well as systemic illness. Eyelash loss/decreases eyelash prominence may also be associated with hair breakage and trauma (i.e. trichotillomania), drug effect (i.e. chemotherapeutic agents) and psychological stress. In the normal population, the most frequent cause of loss of eyelash prominence is attributed to the aging process, as eyelash length (which is a major component of eyelash prominence) decreases with aging.

Based on the development of treatments to promote the prominence of the eyelashes, there is a need for reliable scales to effectively and consistently rate the prominence of the eyelashes. Such scales are important in clinical practice and for clinical trial research. For example, in clinical trial research, objective quantification is critical to measure the efficacy of an investigational treatment by comparing the severity of the condition before treatment to that measured after treatment. For a new treatment to achieve regulatory approval for marketing, its efficacy must be documented in clinical trials. Valid and reliable outcome measures are also important in evidence-based medicine to provide comparisons among similarly designed trials in the literature.

Accordingly, there is a need for reliable and reproducible scales that can be used by clinicians to effectively rate eyelash prominence in deriving appropriate treatments and assessing treatment response for loss of eyelash prominence.

### SUMMARY OF THE INVENTION

Provided herein are scales and methods for performing clinical assessment of a characteristic of an individual. In one embodiment, scales and methods are provided for performing clinical assessment of an individual that includes determining a base clinical assessment for the patient by generating information on a clinical rating scale. Particularly provided are scales and methods utilizing reliable and consistent scales to effectively rate the characteristic of eyelash prominence of an individual.

In one embodiment, a scale is provided for assessing a characteristic of an individual wherein the characteristic is eyelash prominence and wherein the scale comprises rows or columns of photographs corresponding to different grades of eyelash prominence. In some embodiments, the scale has a Kappa score of at least about 0.700 for intra-rater and inter-rater reliability.

In another embodiment, the scale includes word-based descriptions accompanying the rows or columns of photographs corresponding to different prominence categories of the scale.

In another embodiment, the word-based descriptions are followed by at least two explanatory words describing a feature commonly found in the row or column of photographs indicating the prominence category.

In another embodiment, the photographs corresponding to different prominence categories of the scale are presented in columns.

In another embodiment, each column includes six photographs.

In another embodiment, the scale includes four columns of photographs.

In yet another embodiment, at least one photograph on the scale comprises a marking to guide a rater's attention to an area of the photograph.

Another embodiment includes a scale for assessing a characteristic of an individual wherein the characteristic is eyelash prominence and wherein the scale comprises rows or columns of photographs corresponding to different prominence categories of the scale and wherein the scale has a Kappa score of at least 0.700 for intra-rater and inter-rater reliability, the scale includes word-based descriptions accompanying the rows or columns of photographs corresponding to different prominence categories of the scale and wherein the word-based descriptions are followed by at least two explanatory words describing a feature commonly found in the row or column of photographs indicating the prominence category.

Also disclosed herein are methods. In one embodiment the method facilitates the assessment of a characteristic of an individual wherein the characteristic is eyelash prominence and wherein method comprises: providing a scale comprising rows or columns of illustrations, for example, photographs corresponding to different prominence categories of the scale. In some embodiments, the scale has a Kappa score of at least 0.700 for intra-rater and inter-rater reliability.

In another embodiment, the step of providing a scale providing comprises providing word-based descriptions accompanying the rows or columns of photographs corresponding to different prominence categories on the scale.

In another embodiment, the step of providing a scale providing comprises providing word-based descriptions including “none”, “minimal”, “mild”, “moderate”, “marked”, “very marked” or “severe.”

In another embodiment, the step of providing a scale providing comprises providing word-based descriptions followed by at least two explanatory words describing a feature commonly found in the row or column of photographs indicating the prominence category.

In another embodiment, the step of providing a scale providing comprises providing photographs corresponding to different prominence categories of the scale in columns.

In another embodiment, the step of providing a scale comprises providing six photographs in each column.

In another embodiment, the step of providing a scale comprises providing four columns of photographs.

Another embodiment includes a method for assessing a characteristic of an individual wherein the characteristic is eyelash prominence and wherein the method comprises providing a scale comprising rows or columns of photographs corresponding to different prominence categories of the scale and wherein the scale has a Kappa score of at least about 0.700 for intra-rater and inter-rater reliability, the scale includes word-based descriptions accompanying the rows or columns of photographs corresponding to different prominence categories of the scale and wherein the word-based descriptions are followed by at least two explanatory words describing a feature commonly found in the row or column of photographs indicating the severity category.

### BRIEF DESCRIPTION OF THE DRAWINGS

FIG. 1 depicts Grade 1 eyelash prominence.

FIG. 2 depicts Grade 2 eyelash prominence.

FIG. 3 depicts Grade 3 eyelash prominence.

FIG. 4 depicts Grade 4 eyelash prominence.

FIG. 5 shows a scale of the present invention, including four charts each illustrating a different Grade of eyelash prominence.

### DETAILED DESCRIPTION

There is a need for reliable and consistent scales to effectively rate eyelash prominence. Such scales are important in clinical practice, and essential for clinical trial research. Particularly, in clinical trial research, objective quantification is critical to measure the efficacy of an investigational treatment by comparing the severity of the condition before treatment to that measured after treatment.

There are a number of parameters that can be assessed to evaluate the effectiveness of a treatment for improving eyelash prominence. For example, one can assess, without limitation, increased length of lashes, increased numbers of lashes along the normal lash line, increased thickness of lashes, increased luster of lashes, increased pigmentation of lashes and/or increased perpendicular angulation of lashes and lash-like terminal hairs. While all of these parameters can be assessed, they do not provide a reliable and reproducible method to evaluate a static assessment of overall global eyelash health or prominence which is important to patients in the assessment of the effectiveness of a particular treatment.

The present invention provides a Global Eyelash Assessment (GEA) scale for use in the static assessment of overall bilateral upper eyelash prominence. The GEA scale of the invention can use a four-point ordinal scale which includes a brief description of each measure accompanied by representative photographs. This scale provides for a static assessment of overall eyelash prominence, as eyelashes are assessed based on actual appearance on the day of evaluation, without relying on prior memory, perception, or assessment of change as compared to previous assessments.

Using the GEA scale, the overall eyelash prominence of the subject's bilateral upper eyelashes can be assessed by the rater as being, without limitation, one or more of the following four assessments: (1) Grade 1, or Minimal (includes everything up to minimal; i.e., worst possible/none); (2) Grade 2, or Moderate; (3) Grade 3, or Marked; and/or (4) Grade 4, Very Marked (includes very marked and above; i.e., best possible). In determining the appropriate GEA scale score, the rater can evaluate overall eyelash prominence, including elements of length, number, thickness or fullness, luster or color, and perpendicular angulation of both upper eyelashes.

In a specific embodiment of the invention, the scale for assessing eyelash prominence of an individual comprises at least two charts, each chart representing only a single level or grade of eyelash prominence. Each chart may include a plurality of illustrations showing the eye area of at least one human subject taken from at least two different viewpoints. For example, each chart may include an illustration, for example, a photograph or other realistic depiction, of a front view and a superior view of the eye area of a human being having an eyelash prominence of the level or grade being depicted in the chart. In one embodiment, each chart includes illustrations of the eye area of more than one human subject, for example, two or three or more human subjects, each subject having the same level or grade of eyelash prominence being depicted in the chart.

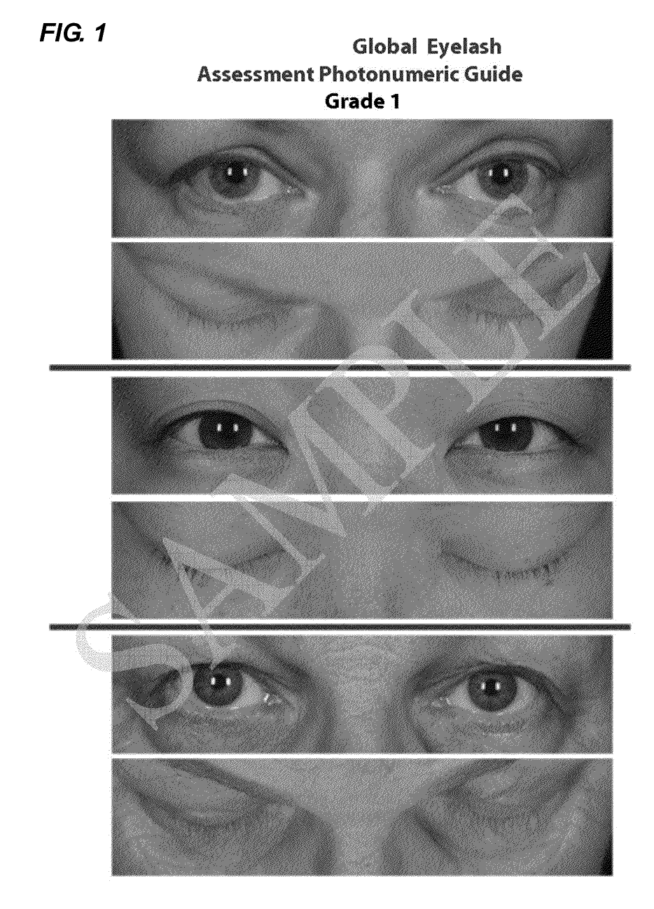

FIGS. 1-4 provide examples of charts including photonumeric guidelines for the rater to consult in deriving a score. That is, the photographic illustrations can provide examples of eyelashes that would be categorized within each eyelash prominence grade. In the provided examples, the photographs are limited to two views or angles (frontal and superior). In certain embodiments, the area of interest can be outlined, shaded or otherwise marked on the photographs. The range of grades on each scale can be intended to represent the full spectrum (minimum to maximum) for eyelash prominence. The photographs can be accompanied by written descriptions.

Particularly, FIG. 1 depicts Grade 1 eyelash prominence according to the GEA scale. As can be seen, in this chart, a column of six photographs is provided. The six photographs represent three examples of eyelashes that correspond to Grade 1 prominence, with a frontal and superior view of each example. Another embodiment includes word-descriptions that describe the photographic examples. Appropriate word-descriptions for Grade 1 eyelash prominence may include, without limitation, Minimal, Worst Possible, None. The chosen word-description should describe a feature commonly found in the corresponding column of photographs.

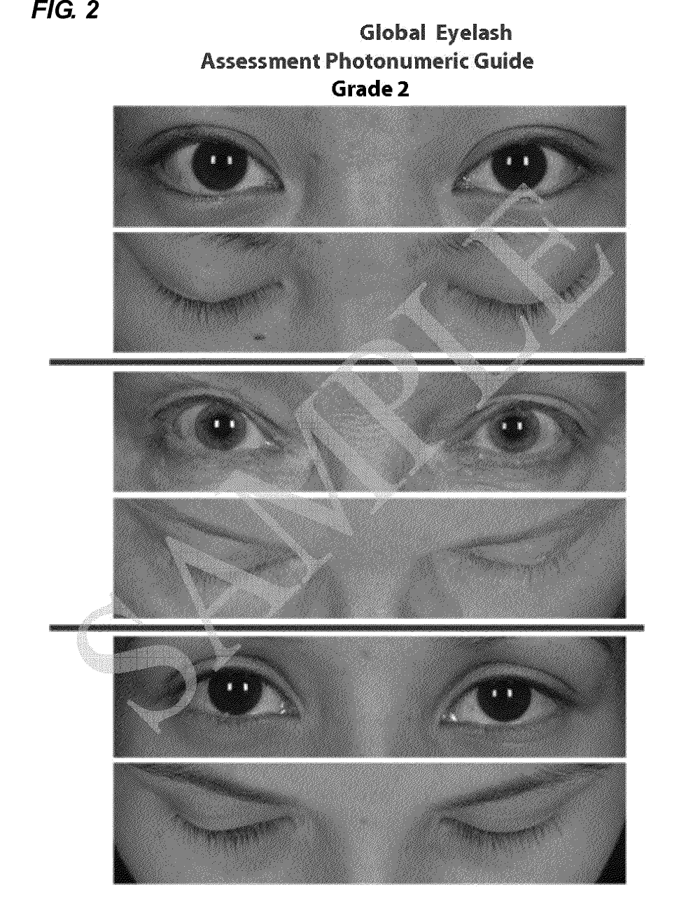

FIG. 2 depicts Grade 2 eyelash prominence according to the GEA scale. As can be seen, in this chart, a column of six photographs is provided. The six photographs represent three examples of eyelashes that correspond to Grade 2 prominence, with a frontal and superior view of each example. Another embodiment includes word-descriptions that describe the photographic examples. Appropriate word-descriptions for Grade 2 eyelash prominence may include, without limitation, Moderate. The chosen word-description should describe a feature commonly found in the corresponding column of photographs.

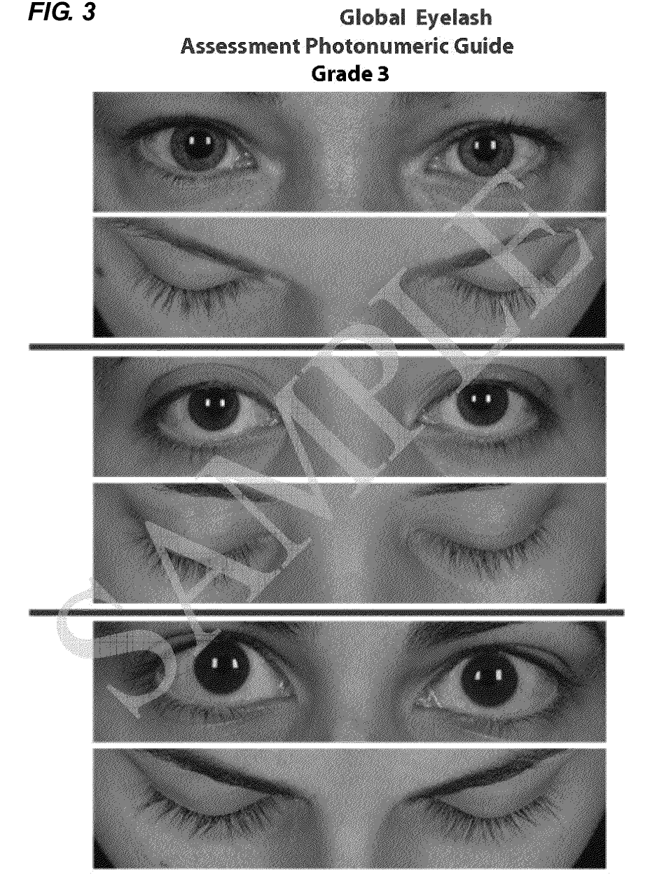

FIG. 3 depicts Grade 3 eyelash prominence according to the GEA scale. As can be seen, in this chart, a column of six photographs is provided. The six photographs represent three examples of eyelashes that correspond to Grade 3 prominence, with a frontal and superior view of each example. Another embodiment includes word-descriptions that describe the photographic examples. Appropriate word-descriptions for Grade 3 eyelash prominence include, without limitation, Marked. The chosen word-description should describe a feature commonly found in the corresponding column of photographs.

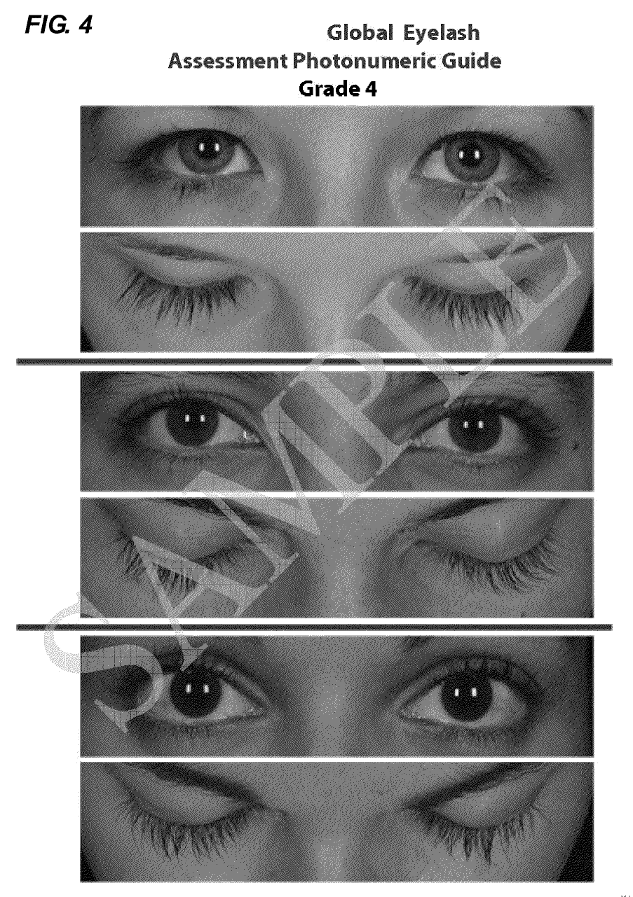

FIG. 4 depicts Grade 4 eyelash prominence according to the GEA scale. As can be seen, in this chart, a column of six photographs is provided. The six photographs represent three examples of eyelashes that correspond to Grade 4 prominence, with a frontal and superior view of each example. Another embodiment includes word-descriptions that describe the photographic examples. Appropriate word-descriptions for Grade 4 eyelash prominence include, without limitation, Very Marked, Best Possible, Excellent. The chosen word-description should describe a feature commonly found in the corresponding column of photographs.

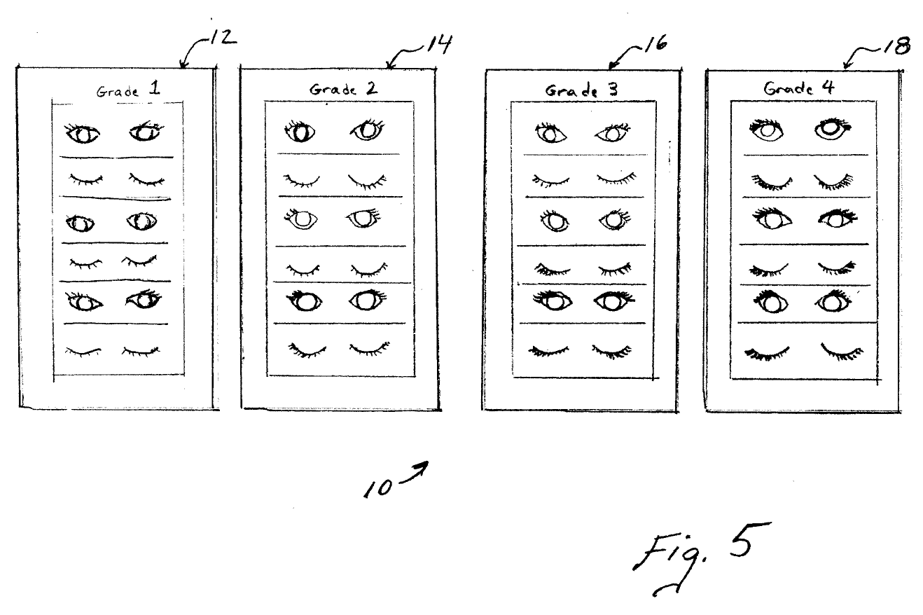

FIG. 5 shows a scale 10 in accordance with an exemplary embodiment of the invention, including four charts, each chart illustrating a different grade of eyelash prominence. More specifically, the scale 10 comprises a first chart 12 showing Grade 1 eyelash prominence, second chart 14 showing Grade 2 eyelash prominence, a third chart 16 showing grade 3 eyelash prominence, and a fourth chart 18 showing Grade 4 eyelash prominence. As shown, each chart 12, 14, 16 and 18 includes illustrations, for example, photographs, specifically, a front view photograph and a superior view photograph, of the eye area of at least two, and in this exemplary embodiment, three, different human subjects having the grade of eyelash prominence depicted on the chart.

The GEA scale produces reliable (reproducible) and consistent ratings for clinician classification of eyelash prominence. The scales described herein have high inter- and intra-rater agreement with their use based on the degree of consistent agreement between assessments performed by multiple clinicians under the same subject presentations, as well as based on the degree of consistent agreement two assessments of the same subject performed at least 1 hour apart, by the same clinician. The kappa statistic can be used as a method for scale validation as it allows the measure of agreement beyond that expected by chance alone. Generally, kappa is calculated by estimating chance agreement and then comparing the observed agreement beyond chance with the maximum possible agreement beyond chance. Kappa estimates from the mKappa.sas macro can be used. Due to their focus these estimates are expected to be lower than weighted kappa estimates. Kappa scores in the range of 0.20 to 0.39 indicate fair agreement, 0.40 to 0.59 indicates moderate agreement, 0.60 to 0.79 indicates substantial agreement, and 0.80 to 1.00 indicates almost perfect agreement. See Landis, JR and Koch, GG, The measurement of observer agreement for categorical data, Biometrics 33:159-174 (1977) which is incorporated by reference in its entirety herein.

The GEA scale can be used to reliably and consistently document the effectiveness of treatments during regular clinical therapy and during clinical trials testing treatment efficacy for therapies or treatments addressing eyelash prominence.

### EXAMPLE

A 52 year old female patient explains to her general practice physician that she has noticed a considerable reduction in the amount and thickness of her eyelashes over the past several years.

The physician examines the patient and suggests that she consider eyelash enhancement therapy. The physician shows the patient a scale such as scale 10 shown in FIG. 5 which comprises four separate charts on individual cards. Together, the physician and the patient examine the patient's eyelashes and compare the patient's eyelashes to the charts. The patient and the physician each individually conclude that the patient has an eyelash prominence of Grade 1.

The patient is prescribed an eyelash enhancement pharmaceutical product (such as Latisse®, available from Allergan, Irvine, Calif.) which she applies to her upper eyelash line daily for one month.

The patient is again examined by the physician and the patient and the physician, after referred to the scale 10, individually conclude that the patient now has an eyelash prominence of Grade 3.

### EXAMPLE

A clinical trial can be conducted to determine the safety and efficiency of a formulation (such as Latisse®) and method for treating hypotrichosis. The clinical trial can measure the efficacy of the formulation and method by comparing the severity of the hypotrichosis before treatment to that measured after treatment.

As a part of the clinical trial, the front view and superior view of the eye area of each clinical trial subject is photographed to establish a baseline Grade. The photographs are compared by researchers to a GEA scale having a Kappa score of at least about 0.700 Kappa intra-rater and inter-rater reliability, such as the scale shown and described elsewhere herein. Each researcher is shown the scale individually and is not provided with each other researchers' conclusion as to Grade. Because of the reliability of the scale, the researchers' conclusions are highly consistent with one another.

Following two months of treatment (i.e. with Latisse®) according to protocol, each subject is again photographed (front view and superior view). These post-treatment photographs are compared to the scale as before. It is evident that a considerable number of the subjects have increased eyelash prominence of at least one Grade level.

Unless otherwise indicated, all numbers expressing quantities or properties and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A scale for assessing eyelash prominence of an individual, the scale comprising:
at least two charts, each chart representing only a single level of eyelash prominence and each chart including a plurality of illustrations showing the eye area of at least one human subject taken from at least two different viewpoints.

2. The scale of claim 1 comprising at least three charts, each chart illustrating a different level of eyelash prominence.

3. The scale of claim 1 comprising four charts, each chart illustrating a different level of eyelash prominence.

4. The scale of claim 1 wherein the illustrations comprise photographs.

5. The scale of claim 1 wherein each chart includes photographs of the eye area of at least two different human subjects.

6. The scale of claim 5 wherein each chart includes a photograph of a front view and a photograph of a superior view of the eye area of each of the human subjects.

7. The scale of claim 1 wherein each chart includes a photograph of a front view and a photograph of a superior view of the eye area of at least three different human subjects.

8. The scale of claim 1 having a Kappa score of at least about 0.700 for intra-rater and inter-rater reliability.

9. A method for assessing eyelash prominence of an individual comprising:
providing a scale comprising illustrations showing the eye area of different human subjects, the illustrations being organized in at least two different charts with each chart showing a particular level of eyelash prominence, and wherein each level of eyelash prominence is represented at least two different human subjects and is taken from two different views of the eye area;
comparing the charts to the eye area of an individual; and
assigning a grade to the individual's eyelash prominence based on the comparison.

10. The method of claim 9 wherein the scale has a Kappa score of at least about 0.700 for intra-rater and inter-rater reliability.

11. The method of claim 9 wherein the two different views of the eye area comprise a frontal view and a superior view.

12. The method of claim 9 wherein the scale comprises at least three charts, wherein each chart illustrating a different level of eyelash prominence.

13. The method of claim 9 comprising four charts, each chart illustrating a different level of eyelash prominence.

14. The method of claim 9 wherein the illustrations comprise photographs.

15. The method of claim 9 wherein each chart includes photographs of the eye area of at least two different human subjects.

16. The method of claim 9 wherein each chart includes a photograph of a front view and a photograph of a superior view of the eye area of each of the human subjects.

17. The method of claim 9 wherein each chart includes a photograph of a front view and a photograph of a superior view of the eye area of at least three different human subjects.
